(19) United States
(12) Patent Application Publication
(10) Pub. No.: US 2007/0259928 A1
(43) Pub. Date: Nov. 8, 2007

(54) Medicinal Composition for Treating Diabetes

(76) Inventors: Taishi Yoshida, Tokyo (JP); Akira Okuno, Tokyo (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/792,879

(22) PCT Filed: Dec. 12, 2005
(86) PCT No.: PCT/JP05/22739

§ 371 Date: Jun. 12, 2007

(30) Foreign Application Priority Data
Dec. 13, 2004 (JP) 2004-359587

(51) Int. Cl. A61K 31/155; A61K 31/41

(52) U.S. Cl. 514/359; 514/636

(57) ABSTRACT

To provide a therapeutic procedure for diabetes mellitus with an excellent efficacy and high safety by possibly reducing side effects. To administer a pharmaceutical composition comprising an FBPase inhibitor at a specified timing.

### TECHNICAL FIELD

The present invention relates to a therapeutic agent containing an FBPase inhibitor as an active ingredient, which is a highly efficacious agent against diabetes mellitus, hyperglycemia, impaired glucose tolerance, obesity, and diabetic complications (preferably diabetes mellitus) and furthermore may reduce adverse events, and to methods for its administration.

In addition, the present invention relates to a therapeutic agent containing an FBPase inhibitor and other antidiabetic agents as active ingredients, which is a highly efficacious agent for diabetes mellitus, hyperglycemia, impaired glucose tolerance, obesity, and diabetic complications (preferably diabetes mellitus) and furthermore may reduce adverse events, and to methods for its administration.

### BACKGROUND ART

FBPase inhibitors are known to reduce blood glucose levels by inhibiting gluconeogenesis in the liver. Thus it is considered that FBPase inhibitors are effective as therapeutic agents against diabetes mellitus, hyperglycemia, impaired glucose tolerance, obesity, and diabetic complications (for example, retinopathy, cataract, nephropathy, peripheral nerve injury, and the like) (for example, see Patent Literatures 1 to 3).

On the other hand, since biguanide preparations exhibit lowering activity on blood glucose levels without weight gain, these preparations are widely used as therapeutic agents for diabetes mellitus in Europe and the USA. Metformin is a currently available biguanide preparation. This agent is administered to patients with diabetes mellitus twice daily. However, it has been known that when biguanide preparations such as metformin and the like are clinically used, they possibly elicit adverse events such as gastrointestinal disorders (for example, nausea, dyspepsia, and the like), and, in rare cases, lactic acidosis could be elicited by accumulation of lactic acid. Thus adequate attentions should be paid when biguanide preparations are clinically used.

In addition, it is well known that regimens containing two or more antidiabetic agents are often prescribed to patients with diabetes mellitus. However, in the conventional combination administration methods, the other antidiabetic agent(s) are generally added to the currently used agents, and only some additive effect is expected. Consequently, there is now a need to develop the most appropriate administration methods of combined antidiabetic agents, that is, efficacious administration methods by which each agent having different modes of action can exert its own merits to the maximum extent and by which at the same time the weak points (side effects and the like) can be suppressed as much as possible.

There have been a number of literatures disclosing lowering effects on blood glucose levels of combined administration of an FBPase inhibitor and other anti-diabetic agents (for example, see Patent Literature 4). However, there is neither description nor suggestion of the specified administration methods discovered by the present invention in these literatures.
[Patent Literature 1] International publication number WO 00/14095
[Patent Literature 2] International publication number WO 01/47935
[Patent Literature 3] International publication number WO 01/66553
[Patent Literature 4] International publication number WO 02/03978

### DISCLOSURE OF THE INVENTION

### OBJECT OF THE INVENTION

The present inventors have diligently conducted research to discover therapeutic procedures with high safety for patients with diabetic mellitus, by exhibiting excellent therapeutic efficacies as well as suppressing side effects (for example, gastrointestinal disorders) and have discovered that high therapeutic efficacies and remarkable suppression of side effects could be obtained by administration of agents containing an FBPase inhibitor at a specified administration timing. Thus the present inventors have completed the present invention.

Furthermore, the present inventors have discovered that combined administration of an FBPase inhibitor and other anti-diabetic agent(s) (preferably a biguanide preparation) at specified administration timings could exert high therapeutic efficacies in terms of blood glucose lowering effects and could suppress side effects remarkably, and consequently, the present inventors have completed the present invention.

[Means to Achieve the Object]

The present invention relates to
(1) a pharmaceutical composition comprising an FBPase inhibitor for prophylaxis or treatment of diabetes mellitus, which is characterized by augmentation of the prophylactic or therapeutic effects on diabetes mellitus when the pharmaceutical composition is administered at an early time of the longest meal interval,
(2) a pharmaceutical composition comprising an FBPase inhibitor for prophylaxis or treatment of diabetes mellitus, which is characterized by augmentation of the prophylactic or therapeutic effects on diabetes mellitus when the pharmaceutical composition is orally administered once daily at an early time of the longest meal interval,
(3) a pharmaceutical composition according to (1) or (2) described above, which is administered at a certain time in the period from after dinner to bedtime,
(4) a pharmaceutical composition according to any one selected from (1) to (3) described above, which is administered at a certain time after dinner,
(5) a pharmaceutical composition according to any one selected from (1) to (4) described above, in which an FBPase inhibitor is contained in an amount of from 1 mg to 400 mg,
(6) a pharmaceutical composition according to any one selected from (1) to (4) described above, in which an FBPase inhibitor is contained in an amount of from 10 mg to 200 mg,
(7) a kit of a pharmaceutical composition for prophylaxis or treatment of diabetes mellitus, in which at least a single unit of an FBPase inhibitor containing an amount of from 1 mg to 400 mg as a single dose is contained with an indication to take the kit orally once daily at an early time in the period from after dinner to bedtime,
(8) a method of treatment for diabetes mellitus, which is characterized by once daily oral administration of a pharmaceutical composition comprising an FBPase inhibitor as an active ingredient, at an early time of the longest meal interval,
(9) a kit of a pharmaceutical composition for the treatment of diabetes mellitus comprising two different active ingredients to be separately administered at an appropriate time interval, which contains (a) a pharmaceutical composition comprising a biguanide preparation as the first component of the active ingredients, and (b) a pharmaceutical composition comprising an FBPase inhibitor as the second component of the active ingredients,
(10) a kit of a pharmaceutical composition for the treatment of diabetes mellitus comprising two different active ingredients to be separately administered at an appropriate time interval to prevent adverse events, which contains (a) a pharmaceutical composition comprising a biguanide preparation as the first component of the active ingredients, and (b) a pharmaceutical composition comprising an FBPase inhibitor as the second component of the active ingredients,
(11) a kit of a pharmaceutical composition for the treatment of diabetes mellitus comprising two different active ingredients to be separately administered at an appropriate time interval to prevent adverse events by reducing the dose of a biguanide. preparation, which contains (a) a pharmaceutical composition comprising a biguanide preparation as the first component of the active ingredients, and (b) a pharmaceutical composition comprising an FBPase inhibitor as the second component of the active ingredients,
(12) a kit of a pharmaceutical composition for the treatment of diabetes mellitus according to any one selected from (8) to (11) described above, wherein it is clearly indicated that (a) the first component of the active ingredients is to be administered at a time when the target patients' glucose uptake into target tissues of the treatment is increased, and (b) the second component of the active ingredients is to be administered at a time when the patients' gluconeogenesis targeted by the treatment is predominantly facilitated,
(13) a kit of a pharmaceutical composition for the treatment of diabetes mellitus according to any one selected from (8) to (11) described above, wherein it is clearly indicated that the second component of the active ingredients is to be administered to the target patients at a time in the period from after dinner to bedtime,
(14) a kit of a pharmaceutical composition for the treatment of diabetes mellitus according to any one selected from (8) to (11) described above, wherein it is clearly indicated that (a) the first component of the active ingredients is to be administered to the target patients at a time in the period from 4:00 to 12:00, while (b) the second component of the active ingredients is to be administered at a time in the period from 17:00 to 1:00,
(15) a kit of a pharmaceutical composition for the treatment of diabetes mellitus according to any one selected from (10) to (14) described above, wherein it is indicated that adverse events of the pharmaceutical composition are gastrointestinal disorders,
(16) a kit of a pharmaceutical composition for the treatment of diabetes mellitus according to any one selected from (10) to (15) described above, wherein it is indicated that an adverse event of the pharmaceutical composition is an increase of lactic acid,
(17) a kit of a pharmaceutical composition for the treatment of diabetes mellitus according to any one selected from (8) to (16) described above, wherein the kit is packed for oral administration,
(18) a kit of a pharmaceutical composition for the treatment of diabetes mellitus according to anyone selected from (8) to (17), wherein the biguanide preparation is metformin, phenformin, or buformin,
(19) a kit of a pharmaceutical composition for the treatment of diabetes mellitus according to any one selected from (8) to (17) described above, wherein the biguanide preparation is metformin,
(20) a kit of a pharmaceutical composition for the treatment of diabetes mellitus according to any one selected from (8) to (19) described above, wherein the FBPase inhibitor is an agent that inhibits human FBPase activity,
(21) a kit of a pharmaceutical composition for the treatment of diabetes mellitus according to any one selected from (8) to (19) described above, wherein the FBPase inhibitor is 2-amino-5-isobutyl-4-{2-[5-(N,N′-bis((S)-1-ethoxycarbonyl)ethyl)phosphonamido]furanyl}thiazole or a pharmacologically acceptable salt thereof,
(22) use of a biguanide preparation and an FBPase inhibitor for the manufacture of a kit of a pharmaceutical composition for the treatment of diabetes mellitus comprising two different active ingredients, (a) a pharmaceutical composition comprising a biguanide preparation as the first component of the active ingredients and (b) a pharmaceutical composition comprising an FBPase inhibitor as the second component of the active ingredients, to be separately administered for treatment of diabetes mellitus at an appropriate time interval,
(23) a therapeutic agent for diabetes mellitus, which is characterized by administering an agent containing a biguanide preparation as an active ingredient and an agent containing an FBPase inhibitor as an active ingredient separately at an appropriate time interval,
(24) a combination of therapeutic agents for diabetes mellitus used in relation to each other, which is characterized by administering (a) a pharmaceutical composition comprising a biguanide preparation as an active component and (b) a pharmaceutical composition comprising an FBPase inhibitor as an active ingredient separately at an appropriate time interval,
(25) a method of treatment for diabetes mellitus, which is characterized by separate administration of (a) a pharmaceutical composition comprising a biguanide preparation as an active ingredient, and (b) a pharmaceutical composition comprising an FBPase inhibitor as an active ingredient at an appropriate time interval, and
(26) a method of treatment for diabetes mellitus, which is characterized by administering (a) a pharmaceutical composition comprising a biguanide preparation as an active ingredient and (b) a pharmaceutical composition comprising an FBPase inhibitor as an active ingredient separately at an appropriate time interval to reduce adverse events caused by the biguanide preparation.

In the present invention, “FBPase inhibitors” are not particularly restricted provided that they inhibit FBPase activity, and they are, for example, phosphoramide ester compounds containing the phosphoramide ester structure as a prodrug moiety which are disclosed in International publication number WO 01/47935 pamphlet, or compounds disclosed in International publication number WO 00/14095 pamphlet, Journal of Medicinal Chemistry, Vol. 45, 3865-3877, 2002, and Bioorganic & Medicinal Chemistry Letters, Vol. 11, 17-21, 2001, preferably phosphoramide ester compounds, and particularly preferably 2-amino-5-isobutyl-4-{2-[5-(N,N′-bis((S)-1-ethoxycarbonyl)ethyl)phosphonamido]furanyl}thiazole and pharmacologically acceptable salts thereof.

In the present invention, “therapeutic agents for diabetes mellitus” are not particularly restricted provided that they are generally prescribed to patients with diabetes mellitus, and they are, for example, insulin preparations, biguanide preparations, insulin secretion enhancers (SU preparations and the like), digesting enzyme inhibitors (α-glucosidase inhibitor and the like), and insulin sensitizers, and the like.

In the present invention, “biguanide preparations” are not particularly restricted provided that they exhibit acceleration of anaerobic glycolysis, potentiation of insulin action at peripheral sites, suppression of glucose uptake from the intestinal canal, and inhibition of gluconeogenesis in the liver, and the like, and they are, for example, 1,1-dimethylbiguanide monohydrochloride (generic name: metformin), phenformin, buformin, and the like, and particularly preferably metformin.

The administration route of therapeutic agents against diabetes mellitus employed in the present invention is generally oral administration. The dosage forms are not particularly restricted provided that they are prepared using conventional formulation techniques, and they are powder, granules, tablets, and capsules.

These preparations are prepared by conventionally known methods using known additive agents in the formulation field of remedies such as excipients, binders, disintegrants, lubricants, dissolution agents, flavors, coating agents, and the like.

For example, when tablets are prepared, inactive ingredients conventionally employed in this field may be widely used. For instance, they may include excipients such as lactose, sucrose, sodium chloride, glucose, urea, starch, calcium carbonate, kaolin, crystalline cellulose, silicic acid, and the like; binders such as water, ethanol, propanol, syrup, glucose solution, starch solution, gelatin solution, carboxymethylcellulose, shellac, methylcellulose, potassium phosphate, polyvinylpyrrolidone, and the like; disintegrants such as dried starch, sodium alginate, agar powder, laminaran powder, sodium hydrogen carbonate, calcium carbonate, polyoxyethylene sorbitan fatty acid esters, sodium lauryl sulfate, stearic acid monoglyceride, starch, lactose, and the like; disintegration inhibitors such as sucrose, stearin, cacao butter, hydrogenated oil, and the like; absorption enhancers such as quaternary ammonium bases, sodium lauryl sulfate, and the like; humectants such as glycerin, starch, and the like; adsorbents such as starch, lactose, kaolin, bentonite, colloidal silicic acid, and the like; and lubricants such as purified talc, stearates, boric acid powder, polyethylene glycol, and the like. In addition, tablets may be conventionally coated when required, for example, they may be prepared as sugar-coated tablets, gelatin-coated tablets, enteric-coated tablets, film-coated tablets, double-coated tablets, and multi-layer tablets.

When pills are prepared, well-known inactive ingredients that are conventionally employed in this field may be widely used. For instance, excipients such as glucose, lactose, starch, cacao butter, hardening vegetable oil, kaolin, talc, and the like; binders such as gum arabic acacia powder, tragacanth powder, gelatin, ethanol, and the like; and disintegrants such as laminaran, agar, and the like may be exemplified. Furthermore, coloring agents, preservatives, perfumes, flavors, sweetening agents, and other remedies may be contained, when required.

The amount of an FBPase inhibitor contained in the above preparations is not particularly restricted and may be selected from a wide range, but it is usually from 1 to 70 weight percent of the total weight of the preparation, and preferably, the appropriate concentration is from 1 to 30 weight percent.

The dose varies depending on a variety of factors such as the symptoms, age, and body weight of the patient, the formulation, and the like. A suitable dosage level is generally from 0.001 mg (preferably 1 mg, and more preferably 10 mg) per day as a lower limit to 2000 mg (preferably 400 mg, and more preferably 200 mg) per day as an upper limit for a human adult.

The amount of a biguanide preparation contained in the above preparations is not particularly restricted and may be selected from a wide range, but it is usually from 1 to 70 weight percent of the total weight of the preparation, and preferably, the appropriate concentration is from 1 to 30 weight percent.

The dose varies depending on a variety of factors such as the symptoms, age, and body weight of the patient, the formulation, and the like. A suitable dosage level is generally from 0.001 mg (preferably 0.01 mg, and more preferably 0.1 mg) per day as a lower limit to 2550 mg (preferably 850 mg, and more preferably 500 mg) per day as an upper limit for a human adult.

### ADVANTAGES OF THE INVENTION

According to the present invention, high therapeutic efficacy with remarkable reduction of side effects, can be achieved by administration of a pharmaceutical composition including an FBPase inhibitor at a specified timing, and therapeutic agents against diabetes mellitus and their administration procedures of the present invention are extremely useful for the treatment of diabetes mellitus.

Furthermore, high therapeutic efficacy and remarkable reduction of side effects can be achieved by co-administration of an FBPase inhibitor and an anti-diabetic agent having a different mechanism of action from that of the FBPase inhibitor at specified timings suitable for each of them, and therapeutic agents against diabetes mellitus and their administration procedures of the present invention are very useful for the treatment of diabetes mellitus.

### BEST MODE FOR CARRYING OUT THE INVENTION

The present invention is illustrated in more detail by the following test examples. However, the present invention is not limited to these examples.

### EXAMPLES

Compound A employed in the Test Examples is a compound disclosed in International-publication number WO 01/47935 pamphlet-and can be manufactured according to methods described in the said publication.

### Test Example 1

### Effects of 2-amino-5-isobutyl-4-{2-[5-(N,N′-bis((S)-1-ethoxycarbonyl)ethyl)phosphonamido]furanyl}thiazole (Compound A) and Metformin on Improvement of Diabetes Mellitus by Administering these Drugs at Specified Timings Suitable for each of Them

Eleven-week old Zucker diabetic fatty (ZDF) rats, that spontaneously developed diabetes mellitus, were grouped into 3 groups (7 rats per group). Feeding was limited to from 17:00 to 9:00, and Compound A and metformin (purchased from Sigma Chemical Co., Ltd.) were repeatedly administered for 6 weeks. Doses of Compound A and metformin were 100-150 mg/kg each once daily, and administered at 9:00 AM (9:00) or 4:00 PM (16:00). In the group administered Compound A at 9:00 AM, metformin was administered at 4:00 PM (AM-Compound A/PM-metformin administered group; Group A), and in the group administered metformin at 9:00 AM, Compound-A was administered at 4:00 PM (AM-metformin/PM-Compound A administered group; Group B). The dose of each compound per day in both groups was fixed at the same. Vehicle alone was administered to the remaining group (control group) twice a day.

After repeated administration of the compounds for 6 weeks, blood sample was collected from the tail vein of each rat, and ratios of glycosylated hemoglobin levels in erythrocytes were determined by DCA2000 manufactured by Bayer Medical Ltd. Glycosylated hemoglobin is non-enzymatically formed by binding of glucose to hemoglobin, and the ratio of glycosylated hemoglobin is increased by sustained hyperglycemia. Thus the glycosylated hemoglobin ratio is widely used as an indicator of a long-term therapy (or control of blood glucose levels) in patients with diabetes mellitus. The average and standard error of the glycosylated hemoglobin ratio determined in each group were calculated and are shown in FIG. 1.

FIG. 1 indicates that the glycosylated hemoglobin ratio in Group A was not significantly different from that in the control group (p=0.0749, Tukey test), although the former value was lower than the latter value. On the other hand, the glycosylated hemoglobin ratio in Group B was significantly lower than that in the control group (p=0.0002, Tukey test) and that in Group A (p=0.0330, Tukey test). From these results, it was clearly demonstrated that the administration procedure of “AM-metformin/PM-Compound A Administration” improved diabetes mellitus more remarkably than the other administration procedure of “AM-Compound A/PM-metformin Administration” in rats.

Thus it was revealed that in the co-administration of the said two agents, their improving efficacies against diabetes mellitus were augmented by administration of these drugs at the specified timings, even though the dose level used in each group was at the same level.

Homeostasis of the blood glucose level is maintained by keeping a balance between glucose production in the liver and glucose uptake into various tissues. Gluconeogenesis contributes to the production of glucose in the liver. In general, it is considered that gluconeogenesis in the liver is potentiated during fasting, and glucose uptake into various tissues is relatively superior to gluconeogenesis during feeding.

An FBPase inhibitor suppresses gluconeogenesis by inhibiting FBPase, which are a rate limiting enzyme of gluconeogenesis in the liver. In addition, it is estimated that metformin facilitates glucose uptake into the muscles, as one of its mechanisms of action, although the mechanisms of action of metformin are not fully understood.

Considering these points, the results obtained in the present Test Example 1 are considered to indicate that gluconeogenesis was inhibited by administration of an FBPase inhibitor during the fasting period when gluconeogenesis is active, and glucose uptake into various tissues was facilitated by administration of a biguanide preparation during the feeding period when glucose uptake is active, and consequently, remarkable improvement against diabetes mellitus was obtained by their individual efficient therapeutic effects.

In the present Test Example 1, combined administration of an FBPase inhibitor in the morning and of a biguanide in the evening resulted in the excellent control of blood glucose level, since daytime and nighttime are fasting and feeding periods, respectively, in rats. On the other hand, in the case of humans, since feeding times are generally opposite to those of rats, excellent improving effects against diabetes mellitus are expected to be brought about by combined administration of a biguanide preparation in the morning (preferably before or soon after breakfast) and that of an FBPase inhibitor in the evening (preferably a time in the period from soon after dinner to before bedtime).

From these considerations, combination administration of an FBPase inhibitor and a biguanide preparation at specified timings most suitable for each of them is considered a very excellent therapeutic procedure for patients with diabetes mellitus, because such administration procedures are able to maintain blood glucose levels at favorable levels and to reduce side effects.

### Test Example 2

### Effects of Feeding on Ameliorating Effects of 2-amino-5-isobutyl-4-{2-[5-(N,N′-bis((S)-1-ethoxycarbonyl)ethyl)phosphonamido]furanyl}thiazole (Compound A) Against Diabetes Mellitus

Thirty-six-week old Goto-Kakizaki (GK) rats (purchased from Charles River Japan, Inc.), that spontaneously developed diabetes mellitus, were grouped into 4 groups such as “non-fasting control group”, “non-fasting and Compound A administered group”, “fasting control group”, and “fasting and Compound A administered group” (6 rats per group).

In Compound A administered groups, Compound A (30 mg/kg) was orally administered, and vehicle alone was administered to two control groups. In addition, rats in two fasting groups were fasted from one day before oral administration of Compound A, and rats in two non-fasting groups were fasted immediately after Compound A was administered [the rats were allowed to take food pellets (FR2, manufactured by Funabashi Farm Co., Ltd.) ad libitum until fasting was started]. Blood sample was collected from the tail vein immediately before and 4 hours after oral administration of Compound A. Blood glucose level was determined by electric potential measurement method based on glucose oxidase immobilized electrode using Glucoloader GXT (A&T Corp.). The blood glucose lowering rate (%) at 4 hours after administration of Compound A against the blood glucose level before administration was calculated in individual rats according to the following equation, and the average blood glucose lowering rate and its standard error in each group are summarized in FIG. 2.
[Blood glucose lowering rate(%)]=100×([blood glucose level at 4 hr after administration]−[blood glucose level before administration])/[blood glucose level before administration]

As shown in FIG. 2, the blood glucose lowering rate in the non-fasting control group was not significantly different from that in the fasting control group (p=0.6439, t-test), and the blood glucose level in the fasting control group was decreased at 4 hours after vehicle administration by almost the same degree compared with that in the non-fasting control group. However, the blood glucose lowering rate at 4 hours after administration of Compound A was significantly reduced in both fasting and non-fasting groups compared with their corresponding control groups (non-fasting group: p=0.0196, fasting group: p=0.0236, t-test), indicating that Compound A lowered the blood glucose level in both fasting and non-fasting conditions.

On the other hand, comparing the average blood glucose lowering rate of the rats at 4 hours after administration of Compound A in “non-fasting and Compound A administration group” with that in the “fasting and Compound A administration group”, the blood glucose lowering rate in the “fasting and Compound A administration group” was significantly lower than that in the “non-fasting and Compound A administration group” (p=0.0158, t-test).

From these results, it was clearly revealed that although Compound A lowered blood glucose levels in both fasted and fed rats, Compound A inhibited blood glucose lowering activity more remarkably when it was administered during a long fasting period.

When these results are applied to human, the longest fasting period (meal interval) in daily life is the period from after dinner to before breakfast. Therefore, it is expected that the improving effects of an FBPase inhibitor are augmented when it is taken from after dinner to bedtime.

Furthermore, based on the results obtained in Test Example 1 and Test Example 2, it is clear that the augmented therapeutic effects obtained by administering Compound A at a suitable time point as described above are not limited only to when Compound A is administered alone, and can be expected when Compound A is administered with other antidiabetic drugs.

### BRIEF DESCRIPTION OF THE DRAWINGS

FIG. 1 is a graph showing effects of 2-amino-5-isobutyl-4-{2-[5-(N,N′-bis((S)-1-ethoxycarbonyl)ethyl)phosphonamido]furanyl}thiazole (Compound A) and metformin on improvement of diabetes mellitus by administering these drugs at specified timings suitable for each of them (Test Example 1).

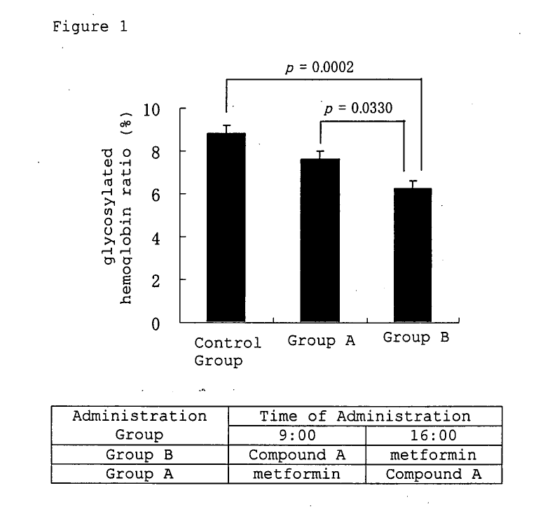

FIG. 2 is a graph showing effects of feeding on ameliorating effects of 2-amino-5-isobutyl-4-{2-[5-(N,N′-bis((S)-1-ethoxycarbonyl)ethyl)phosphonamido]furanyl)thiazole (Compound A) against diabetes mellitus (Test Example 2).

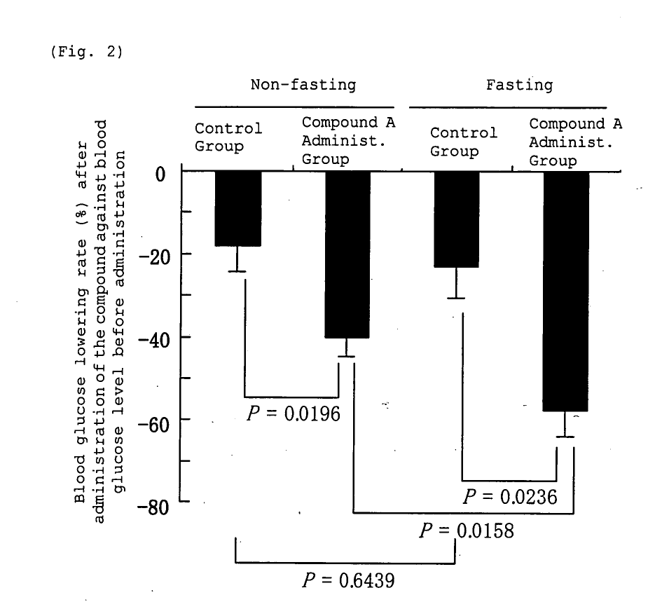

1-6. (canceled)

7. A method for treating diabetes mellitus comprising once daily orally administering to a human a pharmaceutical composition comprising a pharmaceutically effective amount of an FBPase inhibitor as an active ingredient after dinner to before bedtime.

8. A kit for the prophylaxis or treatment of diabetes mellitus in a human, in which at least a single unit of an FBPase inhibitor containing an amount of from 1 mg to 400 mg as a single dose is contained with an indication in the kit to take the FBPase inhibitor orally once daily at an early time in the period between after-dinner and bedtime.

9. A kit comprising two different active ingredients to be separately administered at a certain delay for the treatment of diabetes mellitus in a human, said kit containing (a) a pharmaceutical composition comprising a biguanide as a first component of the active ingredients, and (b) a pharmaceutical composition comprising an FBPase inhibitor as a second component of the active ingredients.

10. A kit comprising two different active ingredients to be separately administered at an appropriate time interval to prevent adverse events in the treatment of diabetes mellitus in a human, said kit containing (a) a pharmaceutical composition comprising a biguanide as a first component of the active ingredients, and (b) a pharmaceutical composition comprising an FBPase inhibitor as a second component of the active ingredients.

11. (canceled)

12. The kit according to any one of claims 9 to 10, wherein it is indicated in the kit that (a) the first component of the active ingredients is to be administered at a time when the human's glucose uptake into target tissues of the treatment is increased, and (b) the second component of the active ingredients is to be administered when the human's gluconeogenesis being targeted by the treatment is predominantly facilitated.

13. The kit according to any one of claims 9 to 10, wherein it is indicated in the kit that the second component of the active ingredients is to be administered at a time in the period from after dinner to bedtime.

14. The kit according to any one of claims 9 to 10, wherein it is indicated in the kit that (a) the first component of the active ingredients is to be administered to the human at a time in the period from 4:00 to 12:00, and (b) the second component of the active ingredients is to be administered at a time in the period from 17:00 to 1:00.

15. The kit according to claim 10, wherein it is indicated in the kit that the use of the kit does not result in gastrointestinal disorders.

16. The kit according to claim 10, wherein it is indicated in the kit that the use of the kit does not result in an increase in lactic acid.

17. (canceled)

18. The kit according to claim 9, wherein the pharmaceutical composition comprises a biguanide which is metformin, phenformin or buformin.

19. The kit according to claim 9, wherein the pharmaceutical composition comprising a biguanide is metformin.

20. The kit according to any one of claims 8 to 9, wherein the FBPase inhibitor is an agent that inhibits human FBPase activity.

21. The kit according to any one of claims 8 to 9, wherein the FBPase inhibitor is 2-amino-5-isobutyl-4-{2-[5-(N,N′-bis((S)-1-ethoxycarbonyl)ethyl)phosphonamido]furanyl}thiazole or a pharmacologically acceptable salt thereof.

22-24. (canceled)

25. A method of treatment for diabetes mellitus comprising separately administering at an appropriate time interval to a human in need thereof (a) a pharmaceutical composition comprising a pharmaceutically effective amount of a biguanide as an active ingredient, and (b) a pharmaceutical composition comprising a pharmaceutically effective amount of an FBPase inhibitor as an active ingredient.

26. A method of treatment for diabetes mellitus comprising separately administering at an appropriate time interval to a human in need thereof (a) a pharmaceutical composition comprising a pharmaceutically effective amount of a biguanide as the active ingredient, and (b) a pharmaceutical composition comprising a pharmaceutically effective amount of an FBPase inhibitor as the active ingredient, to reduce adverse events caused by the biguanide.

27. The method according to claim 25, wherein the biguanide is administered in the morning before or soon after breakfast and the FBPase inhibitor is administered after dinner to before bedtime.

28. The method according to claim 27, wherein the FBPase inhibitor is administered in an amount of 1 mg to 400 mg and the biguanide is administered in an amount of 0.01 mg to 850 mg.

29. The method according to claim 27, wherein the FBPase inhibitor is administered in an amount of 10 mg to 200 mg and the biguanide is administered in an amount of 0.1 mg to 500 mg.

30. The method according to claim 27, wherein the biguanide is administered from 4:00 to 12:00 and the FBPase inhibitor is administered from 17:00 to 1:00.

31. The method according to claim 30, wherein the biguanide is selected from the group consisting of metformin, phenformin and butformin; and the FBPase inhibitor is 2-amino-5-isobutyl-4-{2-[5-(N,N′-bis((S)-1-ethoxycarbonyl)-ethyl)phosphonamido]furanyl}thiazole or a pharmacologically acceptable salt thereof.

32. The method according to claim 31, wherein the biguanide is metformin.
